(19) United States
(12) Patent Application Publication
(10) Pub. No.: US 2003/0175735 A1
(43) Pub. Date: Sep. 18, 2003

(54) Recombinant adenylcyclase and use thereof for screening molecules with proteolytic activity

(76) Inventors: Gouzel Karimova, Paris (FR); Daniel Ladant, Reims (FR); Agnes Ullmann, Paris (FR); Nathalie Dautin, Pamfou (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/204,987

(22) PCT Filed: Feb. 28, 2001
(86) PCT No.: PCT/FR01/00593

(30) Foreign Application Priority Data
Feb. 28, 2000 (FR) 00/02448

(51) Int. Cl. C12Q 1/68; C07H 21/04; C12N 9/22; C12P 21/02; C12N 5/06

(52) U.S. Cl. 435/6; 435/69.1; 435/199; 435/320.1; 435/325; 536/23.2

(57) ABSTRACT

The invention concerns a recombinant adenylcyclase, comprising at least a polypeptide sequence including one or several cleavage site of at least a molecule with site-specific proteolytic activity, said polypeptide sequence being inserted in the catalytic domain of an adenylcyclase while preserving its enzymatic activity. The invention also concerns methods for screening molecules with proteolytic activity using said recombinant adenylcyclase.

The present invention relates to a recombinant adenyl cyclase comprising at least one polypeptide sequence including one or more cleavage sites for at least one molecule with site-specific proteolytic activity, said polypeptide sequence being inserted into the catalytic domain of an adenyl cyclase while at the same time conserving the enzymatic activity thereof. The invention also relates to the DNA fragments encoding such a recombinant adenyl cyclase, and also to the methods for detecting, identifying and/or quantifying proteolytic activity or resistance to inhibitors of proteolytic activity of molecules, using the products defined above. The invention also relates to diagnostic kits for carrying out these methods.

In eukaryotes, as in prokaryotes, proteases are involved in many biological processes. The cascades of activation by proteolysis which lead to clotting and to digestion are now well known, but new phenomena involving these enzymes are regularly being discovered. Some membrane receptors (Protease Activated Receptor: PAR) are specifically activated by proteolysis (Coughlin, 1994, Proc. Natl. Acad. Sci USA, 91, 9200-2). In Bacillus subtilis, the SpoIIGA and SpoIVFB proteases provide conversion of pro-σ^{E} and pro-σ^{K} to σ^{E} and σ^{K}, which are transcription factors essential to sporulation (Hofmeister et al., 1995, Cell, 83, 219-26; Lu et al., 1990, Proc. Natl. Acd. Sci. USA, 87, 9722-6). Caspases, another family of proteases, are involved in apoptosis (Villa et al., 1997, TIBS, 22, 388-93; Steller, 1995, Science, 267, 1445-9) and have an important role in development and homoestasis. Proteases are also involved in certain pathological conditions: Alzheimer's disease is thought to be due to abnormal cleavage of β-amyloids by a serine protease (Selkoe, 1999, Nature, 399, 23-31). In tumors, metalloproteinases (MMPs), by degrading the extra-cellular matrix, allow cells to metastasize (Nagase et al., 1999, J. Biol. Chem., 274, 21491-4). Finally, the protease is an element which is essential to the maturation of many viruses, some of which are responsible for lethal infections (Schwartz et al., 1999, Clin. Diagn. Lab. Immunol., 6, 295-305.)

The identification of these enzymes and the study thereof are therefore necessary both to specify their physiological roles and to develop new therapeutic strategies. The complexity of the conventional methods for characterizing and purifying proteases have led to the development of genetic study systems in Escherichia coli or in yeast. The aim of these systems is to isolate and characterize site-specific proteases. The principle thereof is based either on the inactivation of a reporter enzyme into which the cleavage site specific for the protease studied has been introduced, or on the toxicity of this protease in E. coli.

Sices et al. (1998, Proc. Natl. Acad. Sci. USA, 95, 2828-33) have described a system in which the λ phage repressor is specifically cleaved, leading to passage from the lysogenic state to the lytic state. Specific cleavage sites have also been inserted into E. coli β-galactosidase and thymidylate synthase and into the yeast GAL4 transcriptional activator (Baum et al., 1990, Proc. Natl. Acad. Sci. USA, 87, 5573-7; Kupiec et al., 1996, J. Biol. Chem., 271, 18465-70; Dasmahapatra et al., 1992, Proc. Natl. Acad. Sci. USA, 89, 4159-62). The proteolysis of these proteins was then observed in vivo. Baum et al. (1990, Proc. Natl. Acad. Sci. USA, 87, 10023-7) have shown that overexpression of the HIV protease is toxic in the E. coli strain BL21 (DE3) and have used this property to isolate and study nontoxic mutants of the protease. This toxicity has also been used to screen for protease inhibitors (Buttner et al., 1997, Biochem. Blophys. Res. Commun., 233, 36-8). However, the lack of sensitivity of these systems has limited the use thereof.

This lack of sensitivity stems from the fact that, when the protease studied has weak proteolytic activity, the proteolysis of the target reporter protein is incomplete. In most cases, the residual enzymatic activity of the uncleaved target protein fraction is then sufficient to confer the phenotype associated with the active form of this protein. As a result, it is impossible to phenotypically distinguish the organisms expressing a protease capable of cleaving and inactivating the target reporter protein from those not expressing this specific protease.

Currently, research is being carried out particularly on the development of tests for studying viral proteases, in particular the HIV protease, which is the virus responsible for acquired immunodeficiency syndrome (AIDS).

In fact, the most active molecules currently available for the treatment of HIV infection are HIV protease inhibitors, combined, in the context of triple therapy, with reverse transcriptase inhibitors. The efficacy of these molecules is based on the fact that the protease is essential for multiplication of the virus: HIV is a retrovirus, its capsid contains an RNA which, once introduced into the target cell, is reverse-transcribed by the viral reverse transcriptase. The DNA obtained is integrated into the eukaryotic genome and the genes encoding the structural proteins and the enzymes of the virus are then transcribed and translated into polyproteins by the cellular machinery. The role of the viral protease is to cleave these precursors (gag and pol) into active proteins so as to obtain mature and infectious viruses. The protease is released from the polyproteins by autoproteolysis and then cleaves the other proteins by cleavage of 8 specific sites (p1 to p8 where p5 and p6 are the sites flanking the protease).

During the reverse transcription step, mutations may appear in the sequence of the protease. They are generally silent or lethal, but may sometimes lead to resistance to protease inhibitors (Dulioust et al., 1999, J. Virol., 73, 850-4) and cause treatment failure (Perrin et al., 1998, Science, 280, 1871-3) . This resistance is generally coupled with a decrease in proteolytic activity of the enzyme.

Due to the importance of proteases in general, and of the HIV protease in particular, it is therefore necessary to determine a method which makes it possible to detect the proteolytic activities of molecules, preferably of proteins, these activities preferably being “site-specific”.

The term “site-specific” is intended to mean that the protease recognizes a specific sequence of amino acids in a polypeptide and that it cleaves said polypeptide at a site which depends on the amino acid sequence and on the protease. This site may be located between two amino acids of said specific sequence, but may also be located upstream or downstream of said sequence.

It is essential to introduce improvements into the current systems for detecting proteases. In particular, it is necessary to improve the sensitivity of the systems in order to allow detection of the lower proteolytic activity of some mutated proteases. In the case of HIV, this point is all the more important since its protease has a proteolytic activity which is already relatively low.

In addition, in order for it to be possible to use the tests in the clinic, it is important for them to be easy to carry out, for them to be relatively inexpensive and for it to be possible to obtain the results within a reasonable period of time (a few days). In fact, for studying the HIV protease, a recombinant virus assay (RVA) is currently used, which consists in introducing the gene of the protease of the virus to be studied into a known test virus, and studying said recombinant virus on cell lines. This assay has the drawback that it characterizes only the major population of viruses present in the patient's body, and that it takes a few weeks to obtain the results.

The present invention provides an original solution to the problem of developing assays for detecting molecules with proteolytic activity, by developing a genetic system for detecting such activities, based on the inactivation, by proteolysis, of an adenyl cyclase (or adenylate cyclase), preferably of the adenyl cyclase of Bordetella pertussis.

Adenyl cyclase is an enzyme involved in the synthesis of cyclic AMP (cAMP) from ATP. cAMP is an ubiquitous intracellular mediator which does not, however, appear to be required for the survival or growth of cells, at least in bacteria, under certain growth conditions. In the present invention, cAMP is therefore used as a signaling molecule.

In the context of the present invention, the term “adenyl cyclase” or “adenylate cyclase” is intended to mean any protein having the same biological activity as the adenyl cyclases found in natural organisms, i.e. having the ability to transform ATP into cAMP or, in other words, in accordance with the proteins of the international definition EC 4.6.1.1, or else any enzyme having a similar biological activity derived from an adenylate cyclase. Those skilled in the art are in fact capable, by making certain judicious mutations, or transforming an adenylate cyclase into a guanylate cyclase, i.e. of changing the substrate specificity of the starting protein so that it produces cGMP from GTP (EC 4.6.1.2) (Beuve and Danchin, 1992, J. Mol. Biol., 225, 933-8) and vice versa (Beuve, 1999, Methods, 19, 545-50). Such an enzyme, obtained from an adenylate cyclase, is therefore included in the definition given above.

The system developed in the present invention is based on the proteolytic inactivation of the catalytic domain of said adenyl cyclase (CYA). This domain can complement a bacterial or fungal (including yeast) strain or cell line deficient in endogenous adenyl cyclase (cya⁻) so as to give it back a cya ⁺ phenotype.

Said cya⁺ phenotype is preferably detected by studying a second phenotype of said strain or line, which is more readily detectable, and the appearance of which is linked to enzymatic activity of the adenyl cyclase.

Thus, when a molecule with proteolytic activity is present and active and recognizes the cleavage site inserted into the adenyl cyclase, the latter is cleaved and the complemented strain becomes cya⁻ again.

The term “readily detectable” is intended to mean that it is not necessary to employ excessive means or to use excessive equipment in order to detect the phenotype. In fact, it is possible to detect the cya⁺ phenotype directly, but this requires detecting the formation of cAMP, which can be done by ELISA but requires a certain machine and cannot be performed rapidly. Thus, a “readily detectable” phenotype can preferably be observed macroscopically. For example, it can be directly observable on a Petri dish with a suitable medium.

Some examples of “readily detectable” phenotypes comprise resistance to antibiotics (induced or suppressed by cAMP), catabolism of certain sugars, such as maltose or lactose, or cAMP-induced expression of readily detectable proteins (for example β-galactosidase, luciferase, green fluorescent protein (GFP)). Those skilled in the art are capable of choosing and defining other systems having the same properties.

A subject of the present invention is thus a recombinant adenyl cyclase, characterized in that it comprises at least one polypeptide sequence including one or more cleavage sites for at least one molecule with site-specific proteolytic activity, said polypeptide sequence being inserted into the catalytic domain of an adenyl cyclase while at the same time conserving the enzymatic activity thereof.

In another embodiment of the invention, the inserted polypeptide sequence also comprises a polypeptide sequence corresponding to a molecule with proteolytic activity. In this case, the protease must perform autoproteolysis.

Thus, depending on the embodiment of the invention, the protease of interest is introduced in trans with respect to the sequence containing the cleavage sites (first case), or it is introduced in cis (second case).

Preferably, the polypeptide sequence contains at least one cleavage site specific for a viral protease, preferably the HIV protease, in particular p5 (SEQ ID NO 1) and/or p6 (SEQ ID NO 2).

Another preferred embodiment of the invention relates to a recombinant adenyl cyclase comprising a polypeptide sequence inserted into its catalytic domain while at the same time conserving the enzymatic activity thereof, said polypeptide sequence also containing a viral protease. Preferably, it is the HIV protease bordered by the p5 and p6 cleavage sequences (SEQ ID NO 3).

Any odenyl cyclase with a catalytic site into which it is possible to insert a polypeptide sequence while at the same time conserving the enzymatic activity thereof may be used to implement the invention. However, a preferred adenyl cyclase is the adenyl cyclase of bacteria of the genus Bordetella, in particular B. pertussis, and more especially the catalytic domain of the adenyl cyclase of B. pertussis (SEQ ID NO 4).

Specifically, this domain is composed of two fragments T25 and T18, both necessary for the activity of this adenyl cyclase, and can tolerate considerable insertions (up to 200 residues) between these fragments without its enzymatic activity being affected by this; on the other hand, the two fragments, when dissociated, have no activity. These two fragments correspond to amino acids 1-224 (T25) and 225-400 (T18).

Thus, a most particularly preferred embodiment of the invention consists of the adenyl cyclase of B. pertussis, comprising a polypeptide sequence including one or more cleavage sites for at least one molecule with site-specific proteolytic activity, inserted between residues 224 and 225. However it is clear that the invention cannot be reduced to this site, since it is possible to determine other sites permissive for the insertion of foreign sequences without there being inactivation of the protein. By way of example, mention may be made of residues 137-138, 228-229, 235-236, 317-318, and 384-385 (Ladant et al., 1992, J. Biol Chem., 267, 2244-50). This list is not exhaustive and other sites may also be used for carrying out the invention.

In the present application, it is understood that the terms “residue” and “amino acid” have the same meaning.

The present invention also relates to a polynucleotide (preferably a DNA fragment), characterized in that it encodes an adenyl cyclase according to the present invention, and a vector containing such a DNA fragment or such a polynucleotide or allowing the expression of an adenyl cyclase according to the invention.

The present invention also relates to the use of a recombinant adenyl cyclase according to the invention, as such or expressed by a DNA fragment, polynucleotide or vector, in methods for detecting, identifying and/or quantifying proteolytic activity or resistance to inhibitors of proteolytic activity. Such methods are also part of the invention.

Thus, a method according to the invention, for detecting the proteolytic activity of a molecule, is characterized in that it comprises the steps consisting in:

a. complementing a bacterial or fungal strain or a cell line deficient in endogenous adenyl cyclase with a recombinant adenyl cyclase according to the invention, said bacterial or fungal strain or cell line having a phenotype the expression of which is linked to the enzymatic activity of the adenyl cyclase;

b. bringing said molecule to be tested into contact with said complemented strain or line;

c. culturing said strain or line under conditions for demonstrating the phenotype linked to the activity of the adenyl cyclase;

d. monitoring the expression of said phenotype.

Many bacterial or fungal strains or cell lines deficient in endogenous adenyl cyclase exist. Mention may in particular be made of the E. coli cya⁻ bacterial strains, the bacteria of the genus Salmonella, Saccharomyces yeast strains (Matsumoto et al., 1982, Proc. Natl. Acad. Sci. USA, 79, 2355-9), or the GH1 (Martin et al., 1981, J. Cell. Physiol., 109, 289-97) or lymphoma-derived (Bourne et al., 1975 Science, 187, 750-2) cell lines. Preferably, an E. coli cya⁻ bacterial strain will be used, in particular the DHT1 strain (F⁻, gln V44(AS), recA1 , endA1 , gyrA96 (Nal^{r}), thi1, hsdR17, spoT1, rfbD1, cya-854, ilv-691:: Tn10). This strain, or any mutant of this strain, is also one of the subjects of the invention.

For the purpose of the invention, the term “mutant” of the bacterial strain DHT1 is intended to mean a bacterial strain having a similarity index of at least 90%, preferably 95%, 98% or 99%, as determined, for example, by the RFLP or RAPD method, and having the same phenotype as the DHT1 strain, i.e. cya⁻.

One of the preferred methods for complementing the strain or line used is the introduction of a DNA fragment or a polynucleotide according to the invention. Such a fragment or polynucleotide may be carried by a vector according to the invention, but may also be stably integrated into the chromosome. Those skilled in the art will choose one or other technique depending on the results to be achieved. Preferably, the DNA fragment or polynucleotide is introduced episomally on a vector according to the invention.

The molecule with proteolytic activity is preferably brought into contact by introducing into complemented the strain or line a DNA fragment or polynucleotide encoding said molecule with proteolytic activity and, therefore, by expressing said molecule in said strain.

The readily detectable phenotypes linked to the activity of the adenyl cyclase have already been mentioned. In E. coli, the ability to ferment sugars, such as maltose or lactose, is preferably chosen.

In order to determine the capacity for resistance of the molecule with proteolytic activity, to an inhibitor of proteolytic activity, the method described above will be carried out, also bringing said molecule into contact with said inhibitor in step b.

The level of resistance to the inhibitor can also be measured by quantifying the expression of the phenotype observed. Thus, and depending on the phenotype studied, those skilled in the art will be able to assay the activity of β-galactosidase, the expression of which is naturally controlled by cAMP. They will also be able to assay the activity of other proteins, such as luciferase (in this case, cya⁻ strains will be used with a gene under the control of a cAMP/CAP-dependent promoter), or measure the level of resistance to a given antibiotic, or else the fluorescence emitted when GFP is used. It is also possible to assay the cAMP produced, which gives an exact measurement of the activity of the adenyl cyclase in the host cell.

The methods according to the invention are preferably used to detect the proteolytic activity, and/or the resistance to inhibitors, of the HIV protease.

The methods according to the invention may therefore prove to be extremely precious tools for studying HIV infections, in particular for laboratory research in order to define novel HIV protease-inhibiting molecules, to test the efficacy of the inhibitors during development, or to determine novel mutants, the study of which may help to understand the mechanisms of resistance of the virus.

A subject of the present invention is also the use of an adenyl cyclase, of a DNA fragment or of a vector according to the invention, for producing diagnostic kits for detecting the activity of molecules with proteolytic activity or their resistance to an inhibitor, these molecules being encoded by viruses present in the serum or the cells of a patient.

It is also possible to use the compounds according to the invention for producing a diagnostic kit for quantifying the (molecules with proteolytic activity resistant to an inhibitor/molecules with proteolytic activity not resistant to said inhibitor) ratio in a patient, said molecules with proteolytic activity being encoded by viruses present in the serum or the cells of said patient.

Such diagnostic kits in particular contain

a. a bacterial or fungal strain or a cell line deficient in endogenous adenyl cyclase,

b. a DNA fragment, a purified poly-nucleotide or a vector according to the invention encoding a recombinant adenyl cyclase, into the catalytic site of which are inserted one or more cleavage site(s) corresponding to the molecule with proteolytic activity.

These kits may also optionally contain:

c. specific primers for amplifying the DNA encoding the proteolytic molecule of interest possibly flanked by auto-proteolytic proteolytic sequences, and/or

d. a vector in a configuration such that it is possible to insert therein the DNA encoding the proteolytic molecule of interest amplified using the primers of c., and/or

e. a vector having the same basis as the vector of d., encoding an active proteolytic molecule, in order to have a positive control, and/or

f. culture media which allow growth of the bacterial or fungal strain or cell line of a. and detection of the phenotype associated with the production of cAMP, and/or

g. reagents for quantifying the production of cAMP in the strain or line used, and/or

h. reagents for quantifying the expression of the reporter protein.

Such a diagnostic kit makes it possible to study a protease inserted in trans, since this protease is then introduced on a vector other than that encoding the adenyl cyclase according to the invention. It is therefore then understood that the vector encoding the adenyl cyclase may have already been introduced into the deficient strain, either in episomal form or in a form allowing integration into the genome. The latter case may be particularly preferred, insofar as this then provides a strain initially deficient in endogenous adenyl cyclase (a) stably complemented with an adenyl cyclase according to the invention (b). The use of antibiotics is not then necessary in order to maintain the selection, and the implementation of the method according to the invention is unchanged.

In another scenario, the vector and the strain are provided separately, and the user must transform the strain in order to restore adenyl cyclase activity.

The present invention also covers the diagnostic kits containing:

a. a bacterial or fungal strain or a cell line deficient in endogenous adenyl cyclase,

b. a DNA fragment, a purified poly-nucleotide or a vector encoding an adenyl cyclase, in a configuration such that it is possible to insert the gene encoding the proteolytic molecule of interest, optionally flanked by auto-proteolytic sequences, into the catalytic domain of the adenyl cyclase while at the same time conserving the enzymatic activity thereof,

c. specific primers for amplifying the DNA encoding the proteolytic molecule of interest, optionally flanked by auto-proteolytic sequences, in order to insert it into the DNA fragment of b.

These kits may also optionally contain:

d. a vector having the same basis as the vector of b., encoding an adenyl cyclase, into the catalytic site of which is inserted an active proteolytic molecule, optionally flanked by proteolytic sequences, in order to have a positive control, and/or

e. culture media which allow growth of the bacterial or fungal strain or the cell line of a. and detection of the phenotype associated with the production of cAMP, and/or

f. reagents in order to quantify the production of cAMP in the strain or line used, and/or

g. reagents for quantifying the expression of the reporter protein.

Such a diagnostic kit makes it possible to study the action of the protease in cis, which is particularly advantageous, in particular for detecting resistance to inhibitors, as demonstrated in the examples.

Preferably, these diagnostic kits make it possible to study a viral protease, in particular the HIV protease. In this case, specific primers for amplifying the DNA encoding this protease and the p5 and p6 flanking regions are chosen, in particular the primers of sequences SEQ ID NO 7 and SEQ ID NO 8.

These diagnostic tools make it possible to make an early identification, using the serum of patients suffering from AIDS, of mutants resistant to protease inhibitors. Thus, it is expected that choosing a treatment suitable for a patient as a function of the viral populations which he or she is harboring will make it possible to limit the therapeutic failures.

In fact, the system is simple to use and rapid (PCR on the serum of patients, subcloning in the plasmid which is preferably derived from pUC, and transformation in bacteria, which may be DHT1). It may therefore be hoped that a result will be obtained in only a few days, against 2-3 weeks for the RVA.

In addition, this test limits the handling of the proviral DNA fragment encoding the HIV protease. It is therefore carried out without any risk of contamination and does not require a P3 laboratory, unlike the RVA assay.

Moreover, and as demonstrated in the examples, the invention makes it possible to obtain a very high sensitivity of detection. Specifically, adenyl cyclase is a protein which has a fairly short half-life. In addition, the peptide containing residues 224 and 225 of the AC of B. pertussis is readily accessible to outside proteins, insofar as adenyl cyclase is a relatively flexible protein. The preferred introduction of the proteolysis sites between residues 224 and 225 therefore leads to good exposure of these sites to the protease of interest. This therefore makes it possible to gain sensitivity. Even more sensitivity is gained when use is made of the system in which the protease is inserted in cis and performs autoproteolysis, since, in this case, the cleavage process is intramolecular and there is no competition with other proteins of the strain or line complemented, for example of E. coli.

This genetic system may also be used to carry out large scale screenings in order to search for proteases responsible for cleaving a specific sequence, or the target sequences for a given protease.

Thus, the invention also relates to a method for identifying molecules with site-specific proteolytic activity, in a library of molecules, characterized in that a method as described above is carried out on the various molecules of the library, the adenyl cyclase complementing the bacterial or fungal strain or the cell line being characterized in that it comprises the specific target amino acid sequence for which the possible molecules with proteolytic activity are being sought.

Similarly, the invention relates to a method for identifying the target sequences for a molecule with proteolytic activity, characterized in that a method as described above is carried out on a library of bacterial or fungal strains or cell lines, each one being complemented with an adenyl cyclase according to the invention comprising a different amino acid sequence in order to determine whether this sequence consists of a cleavage site for said molecule with proteolytic activity.

The following examples make it possible to illustrate the invention by developing certain preferred embodiments.

In particular, these examples make it possible to illustrate the various advantages of the invention, in particular the high sensitivity of the methods according to the invention, and the advantages of each system according to the invention (introduction of the molecule with proteolytic activity in cis or in trans).

Based on these examples, those skilled in the art will be capable of improving certain parameters. In particular, all the values given in the examples are done so only by way of indication and should in no way be considered as limiting the invention.

### DESCRIPTION OF THE FIGURES

FIG. 1: Diagrammatic representation of the plasmids used in an embodiment of the invention. plac: lactose operon promoter; T25 and T18: sequences encoding, respectively, the T25 and T18 fragments of the adenyl cyclase of B. pertussis; p5 and p6: sequences encoding the HIV protease cleavage sites; kan^{r}: kanamycin resistance gene; amp^{r}: ampicillin resistance gene. The pKT25 plasmids and derivatives have an origin of replication of the P15A type and are therefore compatible with the pUCVIH plasmids and derivatives (origin of replication: Co1E1).

FIG. 2: Inactivation of the adenyl cyclase in trans by the HIV virus protease. T25 and T18 correspond, respectively, to amino acids 1-224 and 225-400 of AC. p5 is one of the sites specific for cleavage of the HIV protease (upstream of the protease sequence). In A, the recombinant protein ACp5, expressed in E. coli, has a basal, calmodulin-independent, activity which leads to the synthesis of cAMP and to the activation of the lactose and maltose operons. In B, the HIV protease, coexpressed with ACp5, cleaves the protein, which releases the inactive fragments T25 and T18: there is no synthesis of cAMP. In C, the specific inhibited protease cannot inactivate ACp5, which can therefore synthesize cAMP.

FIG. 3: β-Galactosidase activity of the DHT1 bacteria transformed with pKACp5 and pUC19, pKACp5 and pUCVIH or else pKT25 and pUC19. The transformed bacteria are cultured overnight at 30° C. in LB medium+kanamycin+ampicillin, and supplemented with protease inhibitors at the concentrations indicated. The assaying is then carried out as described in example 3.

FIG. 4: Synthesis of cAMP in the cells as a function of the concentration of protease inhibitors in the culture medium. The E. coli DHT1 bacteria were transformed with the plasmid indicated, and then cultured overnight at 30° C. in LB medium+kanamycin+ampicillin, supplemented with protease inhibitors at the concentrations indicated. The cAMP assay is carried out as described in example 4.

FIG. 5: Autoproteolysis of the HIV protease inserted into the adenyl cyclase. T25 and T18 represent, respectively, amino acids 1-224 and 225-400 of the adenyl cyclase of B. pertussis, p5 and p6 are the sites specific for cleavage of the HIV protease. In A, the protease performs autocleavage, which generates the two inactive fragments T25 and T18; the bacteria remain Cya⁻. In B, the protease inhibitors inactivate the protease, which can no longer perform autocleavage; the adenyl cyclase then synthesizes cAMP and restores a Cya⁺ phenotype.

FIG. 6: β-Galactosidase activity of the DHT1 bacteria transformed with pKACp5, pKACPr and pKT25 as a function of the concentration of inhibitor in the culture medium. The transformed bacteria are cultured overnight at 30° C. in LB medium+kanamycin, supplemented with inhibitors at the concentrations indicated. The assay for activity is then carried out as described in example 3.

FIG. 7: Synthesis of cAMP by the DHT1 bacteria transformed with pKACp5, pKACPr and pKT25 as a function of the concentration of inhibitors in the medium. The transformed bacteria are cultured overnight at 30° C. in LB medium+kanamycin, supplemented with inhibitors at increasing concentrations. The assay is then carried out as described in example 4.

### EXAMPLES

### Example 1

Strains and Media

The genetic constructs are prepared in the Escherichia coli strain XL1-Blue (endA1, hsdR17, supE44, thi1,λ⁻, recA1, gyrA96, relA1, Δ(lac-proB)/F⁻, proAB, lac19ZΔM15, Tn10 (tet^{r})) (in particular available from Stratagene) and the activity of the proteins expressed by the plasmids is assayed in the Escherichia coli strain DHT1 (F⁻, gln V44( AS), recA1, endA1, gyrA96 (Nal^{r}), thi1, hsdR17, spoT1, rfbD1, cya-854, ilv-691:: Tn10). This strain was deposited with the CNCM on Jan. 4, 2000, under the item number I-2375.

The bacteria are cultured in Luria-Bertani (LB) liquid or agar (15 g/l agar) medium. Their ability to ferment sugars is tested on McConkey agar medium containing 1% of maltose (Miller, 1972, Experiments in Molecular Genetics, Cold Spring Harbor Lab. Press, Cold Spring Harbor, N.Y.). The antibiotics are used at the following concentrations: ampicillin: 100 μg/ml and kanamycin: 50 μg/ml. The protease inhibitors, indinavir (Crixivan, Merck) and saquinavir (Invirase, Roche) are dissolved, respectively, in ethanol and in water (final concentration 20 mM) and then diluted in the culture media at the concentrations indicated.

### Example 2

Plasmid Constructs

All the plasmids were constructed according to the standard protocols described by Sambrook et al. (1989, Molecular Cloning: a laboratory manual, Cold Spring Harbor Lab. Press, Cold Spring Harbor, N.Y.). The plasmid DNAs were purified using the “Qiagen kit” (Qiagen GmbH, Germany) and hydrolyzed with the appropriate restriction enzymes according to the suppliers' indications (New England Biolabs or Fermentas). The PCR (polymerase chain reaction) conditions are determined as a function of the purine and pyrimidine base composition of the primers (Saiki et al., 1988, Science, 239, 487-91).

The plasmid pUCVIH is a derivative of the plasmid pUC19 (Sambrook et al., 1989) and expresses the wild-type protease of the HIV virus under the control of a lac promoter. To construct it, a PCR was carried out using, as matrix, the proviral DNA of the HIV virus and, as primers, the oligonucleotides A1 (SEQ ID NO 5) and A2 (SEQ ID NO 6).

The PCR product obtained was purified on agarose gel, digested with the BanHI and SalI enzymes and subcloned between the BamHI and SalI sites of pUC19.

The plasmid pUCVIH was deposited with the CNCM on Jan. 4, 2000, under the item number I-2376.

The plasmids pUCB1, pUCB3, pUCV1 and pUCV2 are derivatives of pUC19, which each express a mutant HIV protease. The DNA encoding these proteases was amplified from the serum of patients and used as matrix to carry out a PCR with the primers A1 and A2. The plasmids were then constructed by subcloning the PCR fragments, purified and digested with BamHI and SalI, into pUC19.

The plasmid pKT25 is a derivative of the plasmid pSU (Bartolome et al., 1991, Gene, 102, 75-8) (compatible with the pUC plasmids and derivatives thereof) expressing only the inactive T25 fragment of the adenyl cyclase under the control of a lac promoter.

The plasmid pKAC expresses the whole catalytic domain of the adenyl cyclase. It was constructed by subcloning, into pKT25, the AatII-EcoRI fragment of pCmAHL1 (Karimova et al., 1998, Proc. Natl. Acad. Sci. USA, 95, 5752-6).

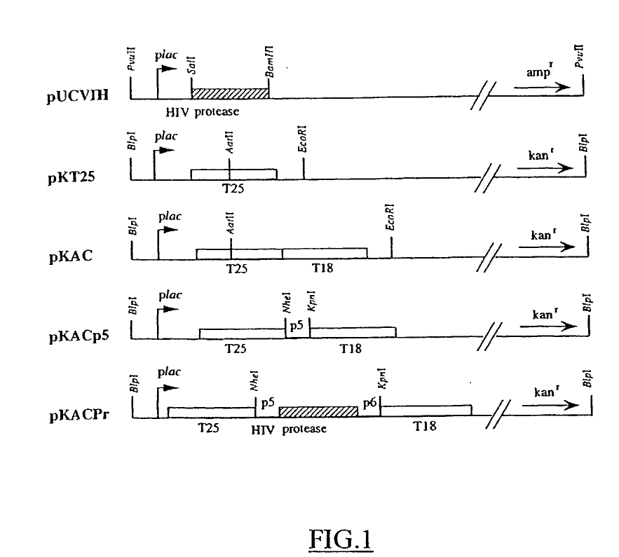

The plasmid pKACPr is a derivative of pSU and expresses, under the control of a lac promoter, the recombinant protein ACp (HIV protease and its two flanking sequences p5 and p6, inserted between amino acids 224 and 225 of the catalytic domain of the adenyl cyclase) (FIG. 1). It was constructed by carrying out a PCR with the primers A3 (SEQ ID NO 7) and A4 (SEQ ID NO 8) on the proviral DNA of the HIV virus. The purified and digested PCR product was then subcloned between the NheI and KpnI sites of pACM224p815A (Karimova et al., 1988, Proc. Natl. Acad. Sci. USA, 95, 12532-7). The plasmid obtained, pACP, was then digested with AatII and EcoRI, and the digestion product was subcloned into pKT25.

Variants of pKACPr, in which the wild-type protease of HIV is replaced with a modified protease (pKACB1, pKACB3, pKACV1 and pKACV2), were constructed. For this, the DNA encoding the mutant proteases was amplified with the primers A3 and A4, and the PCR products, purified and digested with NheI and KpnI, were subcloned between the NheI and KpnI sites of pKACPr.

The plasmid pKACp5 was constructed by hybridizing the two complementary oligonucleotides A5 (SEQ ID NO 9) and A6 (SEQ ID NO 10) and subcloning them between the NheI and KpnI sites of pKACPr. This sequence encodes the p5 polypeptide which is one of the HIV protease cleavage sites.

The plasmids pKACPr and PKACp5 were each deposited with the CNCM on Jan. 4, 2000, under the respective item numbers I-2377 and I-2378.

### Example 3

β-Galactosidase Activity Assay

The β-galactosidase activity is assayed by measuring the hydrolysis of colorless ortho-nitrxophenyl-β-D-galactoside (ONPG), the ortho-nitrophenyl released being colored in basic medium and absorbing at 420 nm (ONP: ε_{mM}=5 at 420 nm and at pH 11).

The bacteria are cultured in LB medium over-night at 30° C. The following day, the suspension is diluted 5-fold in 63B1 medium (Miller, 1972, cf. above), the optical density (OD) at 600 nm of this dilution is measured, and then a drop of toluene and a drop of sodium deoxycholate at 1% are added to 3 ml of this suspension. The tubes are vortexed for 10 seconds and placed at 37° C. for 30 minutes with shaking. This treatment makes the bacterial membranes fragile so that small molecules (ONPG and ONP) diffuse freely.

For the assay, the toluenized suspension is diluted in 1 ml of PM2 buffer (70 mM Na₂HPO₄.12H₂O, 30 mM NaHPO₄.H₂O, 1 mM MgSO₄, 0.2 mM MnSO₄, pH 7, and 100 mM β-mercaptoethanol added extemporaneously). The tubes are placed at 28° C. and, at t=0, the reaction is initiated by adding 0.25 ml of a solution of ONPG (13 mM dissolved in PM2 buffer without β-mercapto-ethanol). When the coloration is sufficient (0.250<OD₄₂₀ <1.6), the reaction is stopped by adding 0.5 ml of 1M Na₂CO₃ (which gives the medium a pH of 11 and inactivates the enzyme).

The OD at 420 nm is read against a control sample (1 ml of PM2 having undergone the same treatment as the other samples).

One unit of β-galactosidase corresponds to 1 nanomole of ONPG hydrolyzed per minute at 28° C. and at pH 7. The number of units per ml is then converted to units per mg of bacterial dry weight, using the OD at 600 nm, in the knowledge that 10⁹ cells correspond to 0.3 mg of bacterial dry weight.

### Example 4

Cyclic AMP Assay

the cAMP produced by the bacteria is measured by indirect ELISA (enzyme linked immunosorbant assay) immunoenzymatic assay, using a rabbit anti-cAMP serum and goat anti-rabbit antibodies coupled to alkaline phosphatase. The alkaline phosphatase substrate used is disodium 5′-para-nitrophenyl phosphate, added at a concentration of 0.5 mg/ml in PA buffer: 100 mM NaCl, 5 mM MgCl₂, 10 mM Tris-HCl, pH 9.5.

After about 1 h at 37° C., reading is performed at λ=405 nm, and the cAMP concentrations are calculated by comparing the ODs obtained for the samples with those of the calibration range. The concentrations (in pmol/ml) are then converted to picomoles per mg of bacterial dry weight (as for the β-galactosidase activity assay).

### Example 5

Inactivation in trans of the Adenyl Cyclase of B. pertussis by the HIV Protease

5.a. Principle of the System

The system is based on the possibility of inserting a polypeptide sequence into the catalytic domain of an adenyl cyclase, without affecting the enzymatic activity thereof.

The adenyl cyclase of B. pertussis can be cleaved by trypsin into two fragments: T25 (amino acids 1-224) and T18 (amino acids 225-400) which, separately, have no catalytic activity (Ladant, 1988, J. Biol. Chem., 263, 2612-8). On the other hand, insertions between amino acids 224 and 225 do not impair its ability to produce cAMP.

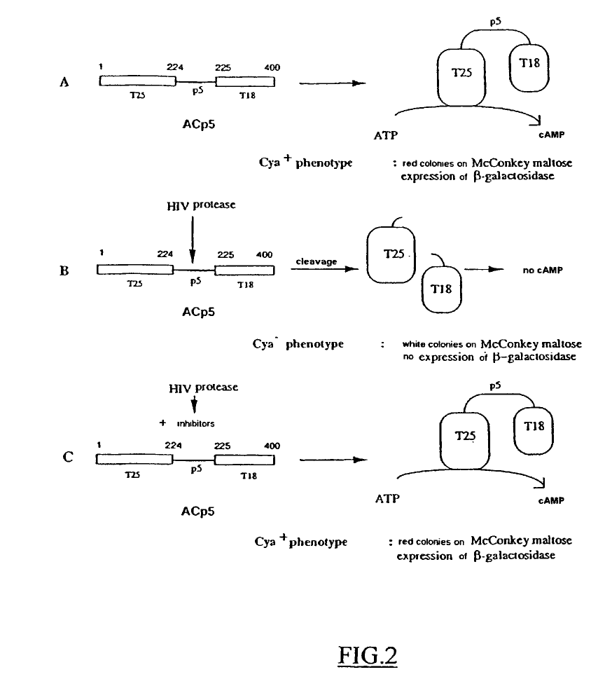

Introducing the specific cleavage site for a given protease between amino acids 224 and 225 of adenyl cyclase does not therefore impair its activity. On the other hand, if this recombinant protein is coexpressed with the corresponding protease, it is specifically cleaved so as to release the two inactive domains T25 and T18. cAMP synthesis does not therefore take place (FIG. 2).

To test the functional activity of the adenyl cyclase of B. pertussis, an E. coli strain deficient in endogenous adenyl cyclase (cya) is used. Specifically, in E. coli, the cAMP which binds to the transcriptional activator CAP (catabolite activator protein) forms a complex which activates many genes, among which are the maltose and lactose operons.

Thus, when the catalytic domain of the adenyl cyclase is expressed in this strain, the production of cAMP allows complementation of the cya characteristic and the bacteria are then capable of fermenting lactose and maltose. T25 and T18 expressed in this same strain as separate entities cannot produce cAMP and the bacteria conserve their cya characteristic.

The ability of the bacteria to ferment sugars can be demonstrated on indicator media (LB-X-Gal or McConkey supplemented with maltose) or on selected media (minimum medium containing lactose or maltose as the only carbon source).

1.b. Development of the System in Vivo

In order to test this system, a recombinant protein according to the invention, ACp5, into which is inserted, between amino acids 224 and 225 of the adenyl cyclase, the p5 cleavage site of the HIV protease, was generated. The plasmid expressing this protein (pKACp5, example 2) was cotransformed into an E. coli cya strain, with a compatible plasmid carrying or not carrying the wild-type HIV protease (pUCVIH or pUC19). The phenotype of the transformants was then observed on McConkey maltose medium containing or not containing protease inhibitors.

The results of these phenotypic tests indicate:

(i) that ACp5 is capable of restoring the Cya⁺ phenotype (red colonies on McConkey maltose medium) when it is expressed in an E. coli cya strain; insertion of the p5 polypeptide between the amino acids 224 and 225 does not therefore impair the adenyl cyclase activity;

(ii) that the DHT1 bacteria cotransformed with pKACp5 and pUCVIH exhibit a Cya⁻ phenotype (white colonies on McConkey maltose medium) in the absence of protease inhibitors; the HIV virus protease is therefore clearly active in E. coli in vivo; it cleaves ACp5, which is then inactivated and can no longer synthesize cAMP. The cleavage of ACp5 by the HIV protease was also shown in vitro;

(iii) that these same transformants, in the presence of protease inhibitors, have a Cya⁺ phenotype; ACp5 is therefore no longer cleaved, which clearly shows that the protease is inhibited by these products;

(iv) finally, to verify that the HIV protease cleaves ACp5 at the p5 sequence and not elsewhere in the protein, the DHT1 bacteria were cotransformed with pUCVIH and pKAC (see example 2, pKAC is a plasmid which expresses the wild-type adenyl cyclase, i.e. without the p5 sequence). The Cya⁺ phenotype of these bacteria shows that the HIV protease is only able to cleave the adenyl cyclase if the latter contains a specific site such as p5.

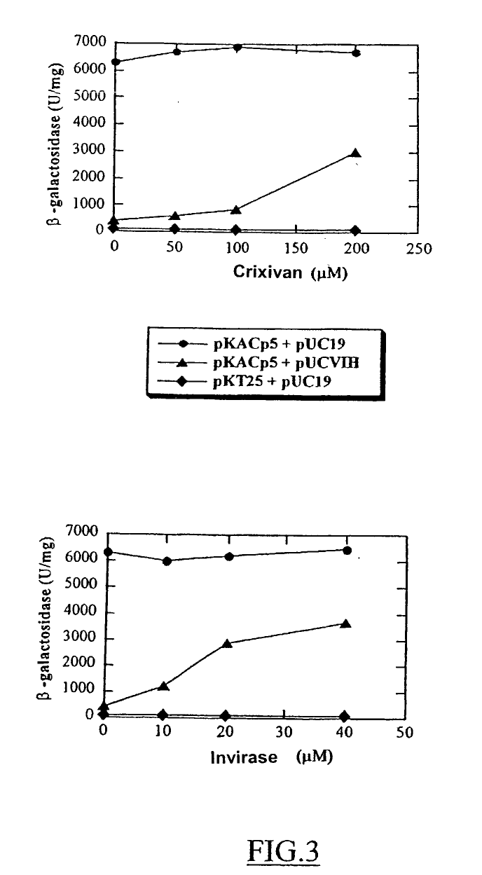

In order to demonstrate the effect of the concentration of inhibitors on the adenyl cyclase activity, the β-galactosidase activity of the cultures in liquid medium of these cells, as a function of the concentration of inhibitors in the medium, was assayed (FIG. 3).

In the case of the DHT1 bacteria transformed with pKACp5 and pUC19, the β-galactosidase activity is high and constant whatever the amount of inhibitors in the medium. With regard to the bacteria cotransformed with pKT25 and pUC19, they have a β-galactosidase activity corresponding to the basic level of the strain which expresses only the T25 fragment (pKT25). For the E. coli DHT1 transformed with pKACp5 and pUCVIH, the increase in the β-galactosidase activity depends on the amount of inhibitors in the medium, which reflects the gradual inhibition of the protease in the cells.

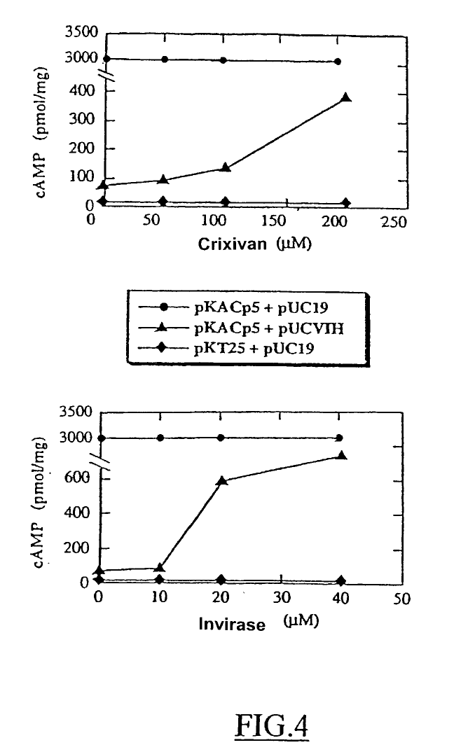

Similarly, the amount of cAMP produced by the DHT1s transformed with pKACPr and pUCVIH increases with the concentration of inhibitors in the medium (FIG. 4), whereas, for the positive control and the negative control (respectively pKACp5+pUC19 and pKT25+pUC19), the cAMP level is constant.

These results show that this genetic system is sensitive enough to allow visualization of the activity of the wild-type HIV protease and to demonstrate inhibition of this activity by specific compounds.

5.c. Detection of HIV Proteases Resistant to Indinavir and to Saquinavir in the System in Vivo

In order to determine whether the method according to the invention is sufficiently sensitive to detect lower activities, mutants resistant to protease inhibitors were studied.

Four clinical isolates (two viruses resistant to protease inhibitors B3 and V2) and two mutant viruses not exhibiting resistance to protease inhibitors (B1 and V1 )) were analyzed. The genotypic and phenotypic characteristics of these four mutants are given in table I.

TABLE 1
Genotypic and phenotypic characteristics of
the HIV protease mutants

| Modified | | Resistance to | Resistance to |
|---|---|---|---|
| proteases | Mutations | saquinavir | indinavir |
| B1 | V771 | 1 X | 1 X |
| B3 | M46I, V77I, V82T | 3 X | 13 X |
| V1 | L63P | 1 X | 1 X |
| V2 | L10I, L63P, L90M | 53 X | 4 X |

The mutations are described relative to the amino acid sequence (1-99) of the reference virus protease (V: Val; I: Ile; M: Met; T: Thr; L: Leu and P: Pro). The level of resistance of the mutants corresponds to the relative increase (compared to the wild-type protease) in the concentration of inhibitors required to inhibit 50% of the viral replication. These data were obtained in in vitro assays (recombinant virus assay).

The DNA encoding the modified proteases was cloned into the vector of pUC19 (example 2) and the plasmids obtained were cotransformed with pKACp5 into the DHT1 strain. The phenotype of the transformed bacteria is observed on McConkey maltose medium containing or not containing protease inhibitors.

Under these conditions, the B1 and V1 proteases behave like the wild-type protease (white colonies in the absence of inhibitors and red colonies with), which is expected since, according to their genotypic and phenotypic characteristics (table 1), they do not exhibit resistance to protease inhibitors.

In the case of the mutant V2, in the presence of indinavir, the phenotype is the same as for the wild-type protease (Cya⁺ using 50 μM of indinavir). In the presence of saquinavir, a higher concentration is needed (20 μM instead of 10 μM) for the bacteria to become Cya⁺. The mutant V2 therefore exhibits resistance to saquinavir (which confirms the data of table 1).

Finally, in the case of the mutant B3, the phenotype of the bacteria is always Cya⁺, including when there are no inhibitors in the medium. This may be explained by the fact that this mutant has lower proteolytic activity than the wild-type protease and that, even though it cleaves a fraction of the adenyl cyclase molecules, there remains a sufficient amount thereof to activate the regulatory cascade resulting in the Cya⁺ phenotype.

The system is therefore sensitive enough to detect the activity of the wild-type protease and of the mutants B1, V1 and V2, but not sufficiently sensitive to detect less active mutants such as B3. This lack of sensitivity of the system may be due to the fact that the cleavage is the consequence of a bimolecular process: specifically, the protease, once synthesized, must, firstly, dimerize and, secondly, interact with its substrate. During this period of time, the uncleaved adenyl cyclase molecules synthesize cAMP which activates the catabolic operons.

In order to improve the sensitivity of the system, another approach, in which the reaction is more direct, was carried out: the whole protease was inserted into the adenyl cyclase, so as to study the autoproteolysis of this chimeric molecule.

### Example 6

Inactivation in cis of the Adenyl Cyclase of B. pertussis. Autoproteolysis of the HIV Protease Inserted into the Adenyl Cyclase

6.a. Principle

This system exploits the particular properties of, firstly, the HIV protease and of, secondly, the adenyl cyclase of B. pertussis. The enzymes and the structural proteins of HIV are synthesized in the form of polyproteins. The maturation of these polyproteins is effected by the protease which can cleave sequences upstream and then downstream of its own sequence (sites p5 and p6). Moreover, adenyl cyclase tolerates considerable insertions (up to 200 residues) between the fragments T25 and T18, without this affecting its enzymatic activity.

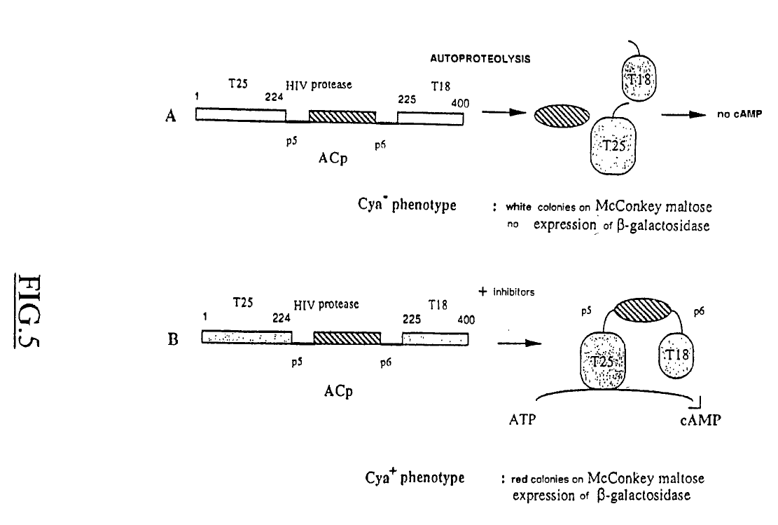

A chimeric protein (ACPr) was therefore constructed (example 2), into which is inserted, between amino acids 224 and 225 of the adenyl cyclase, the HIV protease (99 residues) and its two cleavage sequences p5 and p6 (each one 8 amino acids) . In this case, the wild-type protease performs autoproteolysis, releasing T25 and T18 which, separated, are inactive. Conversely, if the protease is inactive, or in the presence of inhibitors, the autocleavage thereof does not occur and the adenyl cyclase conserves its cAMP synthesis activity and can complement the E. coli cya, which then have a Cya⁺ phenotype (FIG. 5).

6.b. Study of the Wild-Type Protease in the System in Vivo

The phenotype of the DHT1 bacteria transformed with pKACPr or else pKT25 (negative control) or else pKACp5 or pKAC (positive controls) was observed.

The bacteria transformed with pKACp5 or pKAC conserve their Cya⁺ phenotype (red colonies on McConkey maltose) whatever the conditions, since the protease is absent. Conversely, the bacteria expressing T25 alone (DHT1+pKT25) are always Cya⁻ (white colonies) since this fragment is inactive.

Finally, the bacteria transformed with the plasmid pKACPr are Cya⁻ in the absence of inhibitor and Cya⁺ in the presence of saquinavir or indinavir. These results show that the protease is still active when it is inserted into the adenyl cyclase and that it can be inhibited with protease inhibitors.

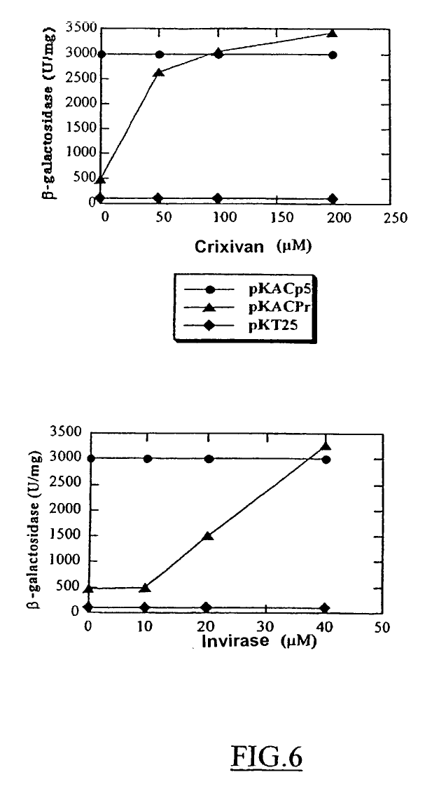

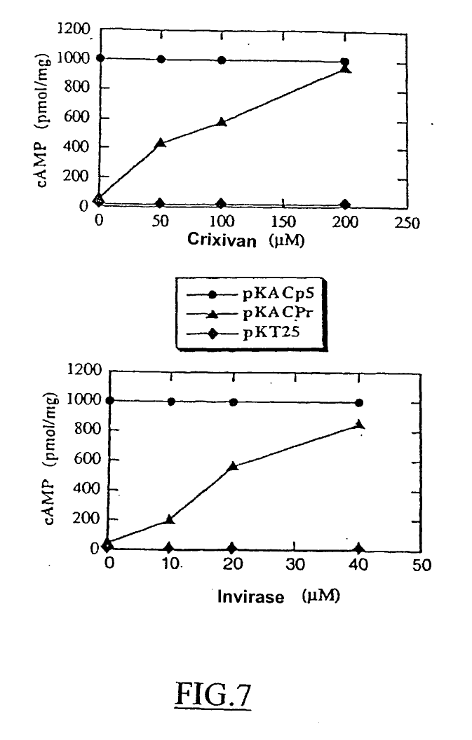

These qualitative results are confirmed by the quantitative data obtained by assaying the cAMP and the β-galactosidase activity in liquid cultures of these cells (FIGS. 6 and 7).

These data indicate that, in the case of the wild-type protease, this system in “cis” is at least as sensitive as that in “trans”: it makes it possible to demonstrate the specific autoproteolysis of the chimeric protein AC/HIV-protease and the inhibition thereof by protease inhibitors.

The sensitivity of this method was then studied in order to determine whether it allows detection of proteases with low activity, as is the case of mutants resistant to inhibitors, in particular the variant B3.

6.c. Detection of HIV Proteases Resistant to Inhibitors in the System in “cis”

For this study, the B1, B3, V1 and V2 proteases are inserted between amino acids 224 and 225 of the adenyl cyclase and the chimeric proteins obtained are expressed in the DHT1 strain (plasmids pKACB1, pKACB3, pKACV1 and pKACV2).

As expected, B1 and V1 behave like the wild-type protease (Cya⁻ phenotype in the absence of inhibitor and Cya⁺ with). The mutant B3 (pKACB3) confers a Cya⁻ phenotype on the bacteria, in the absence of inhibitors. In the presence of indinavir, the bacteria transformed with pKACB3 have a Cya⁻ phenotype up to a concentration of 100 μM (against 50 μM for those transformed with pKACPr), which shows that this protease carries a mutation which makes it resistant to indinavir. In the presence of saquinavir, the DHT1 bacteria transformed with pKACB3 have a Cya⁻ phenotype at 10 μM, whereas those transformed with pKACPr (wild-type protease) are red on this medium. The mutant protease B3 is therefore also resistant to saquinavir.

In the case of mutant protease V2, the system makes it possible to demonstrate the decrease in its sensitivity to saquinavir and to indinavir: the bacteria transformed with pKACV2 are Cya⁻ on medium containing 100 μM indinavir or 20 μM of saquinavir, whereas on these same media, the bacteria transformed with pKACPr give red colonies.

The system in “cis” is therefore much more sensitive than that in which the protease is provided on an independent plasmid; specifically, it detects very low proteolytic activities such as that of the B3 protease and makes it possible to distinguish limited increases in resistance (4X).

### Example 7

Detection of a Minority Population of HIV Proteases Resistant to Inhibitors

This example shows that the invention makes it possible, using a phenotypic assay, to detect, in a population which is mainly sensitive, a minority population of HIV viruses expressing proteases resistant to inhibitors. This method, which can be applied routinely on the serum of patients, makes it possible to detect the emergence of resistance at the early stage of treatment and, optionally, to adjust the treatment as a consequence.

For this, mixtures were prepared containing pKACV2 and pKACPr in variable amounts (1/1, 1/10 and 1/100), and then each of these mixtures was transformed into the DHT1 strain. The phenotype of the transformants is observed on McConkey maltose medium containing 20 μM of saquinavir since this concentration allows the sensitive wild-type protease to be easily distinguished from the saquinavir-resistant protease (V2).

The DHT1 bacteria transformed with pKACPr or pKACV2 are white in the absence of inhibitors. When these same DHT1 are transformed with a mixture of the two plasmids, it is observed that, in the presence of 20 μM of saquinavir, the colonies on the dishes are heterogeneous: white colonies and red colonies are distinguished.

In addition, the ratio of the number of red colonies to the number of white colonies on the dishes corresponds to the relative amounts of pKACPr and pKACV2 transformed in the DHT1 strain. In order to verify that the white colonies harbor pKACV2 and the red ones harbor pKACPr, the plasmids are purified from 4 red colonies and from 4 white colonies, and then digested with EcoRI and KpnI.

The plasmids purified from the red colonies have the same digestion profile as pKACPr (two fragments of 3852 bp and 710 bp), whereas the plasmids derived from the white colonies have the same digestion profile as pKACV2 (they are only digested with KpnI since they do not have EcoRI sites, this having been eliminated by design in order to facilitate distinction between the two plasmids).

The red colonies therefore correctly correspond to bacteria transformed with pKACPr, whereas the white colonies harbor pKACV2. The method described in the present invention therefore makes it possible to phenotypically distinguish proteases resistant to a given inhibitor in a population which contains mainly proteases sensitive to this inhibitor.

Deposition of the Biological Material

The following organisms were deposited on Jan. 4, 2000, with the Collection Nationale de Cultures de Microorganismes (CNCM) [National Collection of Microorganism Cultures], 25 rue du Docteur Roux, 75724 Paris Cedex 15, France, according to the provisions of the Treaty of Budapest.

| | |
|---|---|
| strain DHT1 | item number I-2375 |
| strain XL1/pUCVIH | item number I-2376 |
| strain XL1/pKACPr | item number I-2377 |
| strain XL1/pKACp5 | item number I-2378 |

1. A recombinant adenyl cyclase, characterized in that it comprises at least one polypeptide sequence including one or more cleavage sites for at least one molecule with site-specific proteolytic activity, said polypeptide sequence being inserted into the catalytic domain of an adenyl cyclase while at the same time conserving the enzymatic activity thereof.

2. The adenyl cyclase as claimed in claim 1, characterized in that the inserted polypeptide sequence also comprises a polypeptide sequence corresponding to a molecule with proteolytic activity.

3. The adenyl cyclase as claimed in either of claims 1 and 2, characterized in that the inserted polypeptide sequence contains at least one cleavage site specific for the HIV protease.

4. The adenyl cyclase as claimed in claim 3, characterized in that the cleavage site specific for the HIV protease is the p5 site, comprising the series of amino acids corresponding to SEQ ID NO 1.

5. The adenyl cyclase as claimed in claim 3, characterized in that the inserted polypeptide sequence contains the HIV protease bordered by the p5 and p6 cleavage sequences corresponding to the sequence SEQ ID NO 3.

6. The adenyl cyclase as claimed in one of claims 1 to 5, characterized in that the adenyl cyclase is the adenyl cyclase of Bordetella pertussis.

7. The adenyl cyclase as claimed in claim 6, characterized in that the polypeptide sequence is inserted between amino acids 224 and 225 of the sequence SEQ ID NO 4.

8. A polynucleotide, characterized in that it encodes an adenyl cyclase as claimed in any one of claims 1 to 7.

9. A vector, characterized in that it contains a polynucleotide as claimed in claim 8, or in that it is capable of expressing an adenyl cyclase as claimed in any one of claims 1 to 7.

10. The vector as claimed in claim 9, capable of expressing an adenyl cyclase as claimed in claim 4, characterized in that it is the vector pKACp5 deposited on Jan. 4, 2000, with the CNCM under the item number I-2378.

11. The vector as claimed in claim 9, capable of expressing an adenyl cyclase as claimed in claim 5, characterized in that it is the vector pKACPr deposited on Jan. 4, 2000, with the CNCM under the item number I-2377.

12. A method for detecting the proteolytic activity of a molecule, characterized in that it comprises the steps consisting in:
a. complementing a bacterial or fungal strain or a cell line deficient in endogenous adenyl cyclase with a recombinant adenyl cyclase as claimed in any one of claims 1 to 7, said bacterial or fungal strain or cell line having a phenotype the expression of which is linked to the enzymatic activity of the adenyl cyclase;
b. bringing said molecule to be tested into contact with said complemented strain or line;
c. culturing said strain or line under conditions for demonstrating the phenotype linked to the activity of the adenyl cyclase;
d. monitoring the expression of said phenotype.

13. The method as claimed in claim 12, characterized in that the bacterial or fungal strain or cell line deficient in endogenous adenyl cyclase is complemented by introduction of a polynucleotide as claimed in claim 8, or of a vector as claimed in one of claims 9 to 11.

14. The method as claimed in either of claims 12 and 13, characterized in that the bacterial strain deficient in endogenous adenyl cyclase is Escherichia coli.

15. The method as claimed in one of claims 12 to 14, characterized in that the phenotype the expression of which is linked to the enzymatic activity of the adenyl cyclase is the ability to ferment lactose or maltose.

16. The method as claimed in one of claims 12 to 14, characterized in that the phenotype the expression of which is linked to the enzymatic activity of the adenyl cyclase is the ability to be resistant to an antibiotic.

17. The method as claimed in one of claims 12 to 14, characterized in that the phenotype the expression of which is linked to the enzymatic activity of the adenyl cyclase is the ability to express a readily detectable protein, in particular luciferase or GFP.

18. A method for detecting the resistance of a molecule with proteolytic activity, to an inhibitor, characterized in that it comprises the steps of a method as claimed in any one of claims 12 to 17, and in that it also comprises bringing said molecule into contact with said inhibitor in step b.

19. The method as claimed in claim 18, characterized in that the level of said resistance is also measured by quantifying the expression of the phenotype studied.

20. The method as claimed in either of claims 18 and 19, characterized in that said molecule with proteolytic activity is the HIV protease.

21. A diagnostic kit for detecting molecules with proteolytic activity, characterized in that it contains:
a. a bacterial or fungal strain or a cell line deficient in endogenous adenyl cyclase,
b. a DNA fragment, a purified polynucleotide or a vector encoding a recombinant adenyl cyclase, into the catalytic site of which are inserted one or more cleavage site(s) corresponding to the molecule with proteolytic activity.

22. A diagnostic kit for detecting molecules with proteolytic activity, characterized in that it contains:
a. a bacterial or fungal strain or a cell line deficient in endogenous adenyl cyclase,
b. a DNA fragment, a purified polynucleotide or a vector encoding an adenyl cyclase, in a configuration such that it is possible to insert the gene encoding the proteolytic molecule of interest, optionally flanked by auto-proteolytic sequences, into the catalytic domain of the adenyl cyclase while at the same time conserving the enzymatic activity thereof,
c. specific primers for amplifying the DNA encoding the proteolytic molecule of interest, optionally flanked by auto-proteolytic sequences, in order to insert it into the DNA fragment of b.

23. The use of an adenyl cyclase as claimed in any one of claims 1 to 7, of a polynucleotide as claimed in claim 8, or of a vector as claimed in one of claims 9 to 11, for producing a diagnostic kit for detecting the activity of molecules with proteolytic activity or their resistance to an inhibitor, these molecules being encoded by viruses present in the serum or the cells of a patient.

24. The use of an adenyl cyclase as claimed in any one of claims 1 to 7, of a polynucleotide as claimed in claim 8, or of a vector as claimed in one of claims 9 to 11, for producing a diagnostic kit for quantifying the (molecules with proteolytic activity resistant to an inhibitor/molecules with proteolytic activity not resistant to said inhibitor) ratio in a patient, said molecules with proteolytic activity being present in the serum or the cells of said patient.

25. The use as claimed in either of claims 23 and 24, characterized in that the molecule with proteolytic activity is the HIV protease.

26. A method for identifying molecules with site-specific proteolytic activity, in a library of molecules, characterized in that a method as claimed in one of claims 12 to 17 is carried out on the various molecules of the library, the adenyl cyclase complementing the bacterial strain comprising the specific target amino acid sequence for which the possible molecules with proteolytic activity are being sought.

27. A method for identifying the target sequences for a molecule with proteolytic activity, characterized in that a method as claimed in one of claims 12 to 17 is carried out on a library of bacterial or fungal strains or cell lines, each one being complemented with an adenyl cyclase as claimed in one of claims 1 to 7 comprising a different amino acid sequence in order to determine whether this sequence consists of a cleavage site for said molecule with proteolytic activity.

28. An Escherichia coli bacterial strain deficient in endogenous adenyl cyclase, characterized in that it is the DHT1 strain deposited on Jan. 4, 2000, with the CNCM under the item number I-2375, or a mutant of this strain.

29. A vector encoding the HIV protease, characterized in that it is the vector pUCVIH deposited on Jan. 4, 2000, with the CNCM under the item number I-2376.


SEQUENCE LISTING (10 sequences)

SEQ ID NO: 1 - protein, 12 aa, Human immunodeficiency virus
  HIV protease cleavage site p5.
TVSFNFPQITLW

SEQ ID NO: 2 - protein, 12 aa, Human immunodeficiency virus
  HIV protease cleavage site p6.
GCTLNFPISPIE

SEQ ID NO: 3 - protein, 150 aa, Human immunodeficiency virus
  HIV protease and its flanking sequences.
GRDNNSLSEAGADRQGTVSFNFPQITLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEE

SEQ ID NO: 4 - protein, 400 aa, Bordetella pertussis
  Catalytic site of the adenyl cyclase of Bortella pertussis
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQ

SEQ ID NO: 5 - DNA, 33 bp, Artificial sequence
  Primer A1 for amplifying the HIV protease
gcggtcgactcatatgggactgtatcctttaac

SEQ ID NO: 6 - DNA, 23 bp, Artificial sequence
  Primer A2 for amplifying the HIV protease.
cgcggatccagtttcaataggac

SEQ ID NO: 7 - DNA, 27 bp, Artificial sequence
  Primer A3 for amplifying the HIV protease and its p5 and p6 regions.
ggggctagcggtagagacaacaactcc

SEQ ID NO: 8 - DNA, 26 bp, Artificial sequence
  Primer A4 for amplifying the HIV protease and the p5 and p6 regions.
cccggtaccttcttctgtcaatggcc

SEQ ID NO: 9 - DNA, 42 bp, Artificial sequence
  Oligonucleotide for constructing the plasmid pKACp5.
gtaccccaaagagtgatctgagggaagttaaaggatacagtg

SEQ ID NO: 10 - DNA, 42 bp, Artificial sequence
  Oligonucleotide for constructing the plasmid pKACp5.
ctagcactgtatcctttaacttccctcagatcactctttggg